(19) United States
(12) Patent Application Publication
(10) Pub. No.: US 2011/0160856 A1
(43) Pub. Date: Jun. 30, 2011

(54) Systems and Methods for Zipknot ACL Fixation

(75) Inventors: M. Mary Sinnott, Logan, UT (US); Daniel F. Justin, Logan, UT (US); Andrew Fauth, River Heights, UT (US); Chad Lewis, Layton, UT (US); Stuart Goble, Logan, UT (US); Karen E. Mohr, Salt Lake City, UT (US)

(73) Assignee: MEDICINELODGE, INC. DBA IMDS CO-INNOVATION, Logan, UT (US)

(21) Appl. No.: 12/828,856

(22) Filed: Jul. 1, 2010

Related U.S. Application Data
(60) Provisional application No. 61/222,574, filed on Jul. 2, 2009.
(60) Provisional application No. 61/333,363, filed on May 11, 2010.
(60) Provisional application No. 61/333,548, filed on May 11, 2010.

(51) Int. Cl. A61F 2/08

(52) U.S. Cl. 623/13.14

(57) ABSTRACT

A system and method for securing an ACL graft. The system may comprise a line routed through a plate, the plate may comprise an elongated body with a plurality of passageways, and a dogbone feature on one end. The line is routed such to create at least one one-way slide so no knots are required. The line may comprise an adjustable loop that receives the graft. The adjustable loop is adjustable through the one-way slide feature of the line routed around and through the plate. The plate may be configured to pass through a bone tunnel with the use of filaments routed through the passageways of the plate or around the dogbone feature of the plate. The filaments may be used to toggle the plate after passage through the bone tunnel to prevent withdrawal back through the bone tunnel because the plate contact area is larger the bone tunnel area.

### CROSS-REFERENCE TO RELATED APPLICATIONS

This application claims the benefit of the following, which is incorporated herein by reference:

U.S. Provisional Patent Application No. 61/222,574 filed 2 Jul. 2009, which carries Applicants' docket no. MLI-77 PROV, and is entitled ZIP KNOT ACL FIXATION BUTTON;

U.S. Provisional Patent Application No. 61/333,363 filed 11 May 2010, which carries Applicants' docket no. MLI-84 PROV, and is entitled ZIP KNOT ACL FIXATION BUTTON; and

U.S. Provisional Patent Application No. 61/333,548 filed 11 May 2010, which carries Applicants' docket no. MLI-85 PROV, and is entitled ZIP KNOT ACL FIXATION BUTTON.

The following are incorporated herein by reference:

U.S. patent application Ser. No. 11/001,866 filed 1 Dec. 2004, now U.S. Pat. No. 7,594,923, which carries Applicants' docket no. MLI-17, and is entitled LINE LOCK SUTURE ATTACHMENT SYSTEMS AND METHODS;

U.S. patent application Ser. No. 10/936,376 filed 7 Sep. 2004, now U.S. Pat. No. 7,566,339, which carries Applicants' docket no. MLI-15, and is entitled ADJUSTABLE LINE LOCKS AND METHODS;

U.S. patent application Ser. No. 10/936,376 filed 11 Jun. 2003, now U.S. Pat. No. 7,150,757, which carries Applicants' docket no. 13447.35, and is entitled ADJUSTABLE LINE LOCKS AND METHODS;

U.S. patent application Ser. No. 11/112,814 filed 21 Apr. 2005, now U.S. Pat. No. 7,641,694, which carries Applicants' docket no. MLI-23, and is entitled LINE LOCK GRAFT RETENTION SYSTEM AND METHOD;

U.S. patent application Ser. No. 11/125,885 filed 8 May 2005, now issued, which carries Applicants' docket no. MLI-32, and is entitled COMPACT LINE LOCKS AND METHODS; and

U.S. patent application Ser. No. 11/142,933 filed 2 Jun. 2005, which carries Applicants' docket no. MLI-33, and is entitled BONE IMPLANTS WITH INTEGRATED LINE LOCKS.

### BACKGROUND OF THE INVENTION

1. The Field of the Invention

The present invention relates generally to anterior cruciate ligament (ACL) repair and the fixation of an ACL graft on the cortical side of the bone. The present invention may also be used for other suspensory fixation applications such as bone/tendon or bone/ligament attachment.

2. The Relevant Technology

Currently ACL repair requires cortical fixation using some type of fixation device that can retain a graft ligament passed through a bone tunnel while maintaining fixation on the cortical side of the bone. Currently there are buttons on the market that allow for fixation without passing through the bone tunnel. Knots tied on, around or through the button are used to hold the graft and the button in place. However, knots are known for reducing the strength of the fixation.

In addition, knots do not offer the amount of tension typically desired by physicians because in tying the knot tension is often relinquished in order to achieve a completed knot. Numerous devices have been developed to eliminate the need to tie knots as a way of securing a line. The devices that accomplish the same function as a knot, which is in part to secure a line to retain tension in a portion of the line, are typically referred to as line locks. These line locks can be used as a one-way directional slide to increase tension in a line without relinquishing that tension to tie a knot.

Current ACL repair systems will engage a graft and then fix the graft using knots tied to a body on the cortical side of the bone. Physicians either have to fix the graft using cord or line prior to passage through the bone tunnel and then readjust the tension, or pass the lines and cords through the bone tunnel without tension and then adjust the tension after pass through, again, tying knots to fix the graft to the cortical fixation device.

In addition currently physicians must choose a proper suture length and bight length of a sling to hold the graft. In this case the surgeon must have multiple sutures with multiple bight lengths available in the operating room (OR) and if the improper length is chosen first then the surgeon will be required to find a different suture length and bight length leading to more guess work and longer surgery times.

As the above described techniques illustrate, the existing systems and procedures for ACL repair may not be as effective as desired.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a perspective view of a device with a neck and head of the plate, a plurality of passageways through a body of the plate, a line routed through the plurality of passageways, a primary filament and a secondary filament;

FIG. 2 illustrates a side view of the device of FIG. 1 with the line routed through the plurality and a primary and secondary filament;

FIG. 3 illustrates a perspective view of the device of FIG. 1 with a plate, the plate with a head, neck, body and a plurality of passageways and the line routed through the plurality of passageways with a primary and secondary filament, the secondary filament routed differently than in FIGS. 1 and 2;

FIG. 4 illustrates perspective view of the device of FIG. 1 with a plate, the plate with a head, neck, body and a plurality of passageways and the line routed through the plurality of passageways with a primary and secondary filament, the secondary filament routed differently than in FIGS. 1 and 3;

FIG. 5 illustrates a perspective view of the device of FIG. 1 with a plate, the plate with a head, neck, body and a plurality of passageways and the line routed through the plurality of passageways with a primary and secondary filament, the secondary filament routed differently than in FIGS. 1, 3 and 4;

FIG. 6 illustrates a perspective view of the device of FIG. 1 with a plate, the plate with a head, neck, body and a plurality of passageways and the line routed through the plurality of passageways with a only a primary filament routed differently than in FIGS. 1, 3, 4 and 5;

FIG. 7 illustrates a perspective view of an alternate embodiment of FIG. 1 with a neck, a head and a body of the plate, the body with a plurality of passageways and a line routed through the plurality of passageways, and a plurality of grooves, the line comprising a first loop or eyelet at one end;

FIG. 8 illustrates a perspective view of an alternate embodiment of the device of FIG. 1, with a plate having a head, a neck and a body, the body with a plurality of passageways and a line routed through the plurality of passageways, and a crimp on the neck of the plate;

FIG. 9 illustrates a perspective view of an alternate embodiment of the device of FIG. 1, with a plate having a head, a neck and a body, the body with a plurality of passageways and a line routed through the plurality of passageways, and hole passing longitudinally through the neck from a lateral passageway with an opening allowing a filament to be looped around the line and two pins holding the line in place across the opening;

FIG. 10 illustrates a perspective view of an alternate embodiment of the device of FIG. 1, with a plate having a head, a neck and a body, the body with a plurality of passageways and a line routed through the plurality of passageways, one of the passageways on the neck of the plate and a loop of the line wrapped around the neck and a filament passing through the neck passageway and looped around the line so the filament is retained by the line;

FIG. 11 illustrates a perspective view of an alternate embodiment of the plate of FIGS. 1-7, with the plate having a head, a neck and a body, the body with a plurality of passageways for receiving at least one line, without the line depicted;

FIG. 12 illustrates a bottom perspective view of the plate of FIG. 11, with the plate having a head, a neck and a body, the body with a plurality of passageways for receiving at least one line;

FIG. 13 illustrates a perspective view of an alternate embodiment of the plate of FIGS. 1-7, with the plate having a head, a neck and a body, the body with a plurality of passageways for receiving at least one line, without the line depicted;

FIG. 14 illustrates a bottom perspective view of the plate of FIG. 13, with the plate having a head, a neck and a body, the body with a plurality of passageways for receiving at least one line and curved ends of the passageways adjacent to one another;

FIG. 15 illustrates a bottom perspective view of the plate of FIG. 13, with the plate having a head, a neck and a body, the body with a plurality of passageways for receiving at least one line and curved ends of the passageways separated by a portion of the body of the plate;

FIG. 16 illustrates a perspective view of an alternate embodiment of the plate of FIGS. 1-7, with the plate having a head, a neck and a body, the body with a plurality of passageways for receiving at least one line, without the line depicted;

FIG. 17 illustrates a bottom perspective view of the plate of FIG. 16, with the plate having a head, a neck and a body, the body with a plurality of passageways for receiving at least one line;

FIG. 18 illustrates a perspective view of an alternate embodiment of the plate of FIGS. 1-7, with the plate having a head, a neck, resembling a single post, and a body, the body with a plurality of passageways for receiving at least one line, without the line depicted;

FIG. 19 illustrates a perspective view of an alternate embodiment of the plate of FIGS. 1-7, with the plate having a head, a neck and a body, the body with a plurality of passageways for receiving at least one line, and at least one deep groove extending from the end of the head to at least one of the plurality of passageways for receiving a line, without the line depicted;

FIG. 20 illustrates a perspective view of an alternate embodiment of the plate of FIGS. 1-7, with the plate having a head, a neck and a body, the body with a plurality of passageways for receiving at least one line, and a cut out extending from the head toward the neck for receiving a line, without the line depicted;

### DETAILED DESCRIPTION OF THE PREFERRED EMBODIMENTS

The present device provides a means for “locking” a line or suture at a length without using a knot, such knots capable of reducing its strength. The invention allows for adjustment of the line/suture/sling length on the back table, reducing the need for multiple sizes or parts. Because of the adjustable nature the tolerance/error band on the line length is lower than competitive devices that use a fixed size and/or length line. Parts of this device may include a zip knot or “wrap” along with plate geometry and hole patterns for routing of a line. The device may also include a loop/cinch with a first loop or lead loop. Other features may include adjustability of a single free end or multiple free ends. Furthermore a first or primary filament and second or secondary filament configurations may be used to flip the plate to secure the device against a bone.

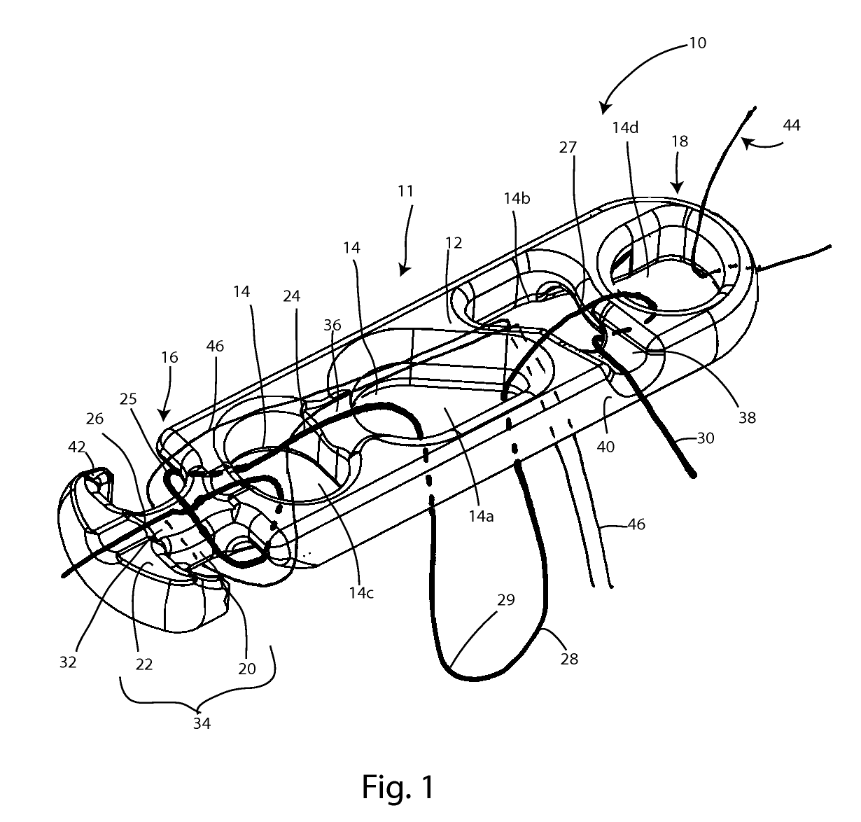

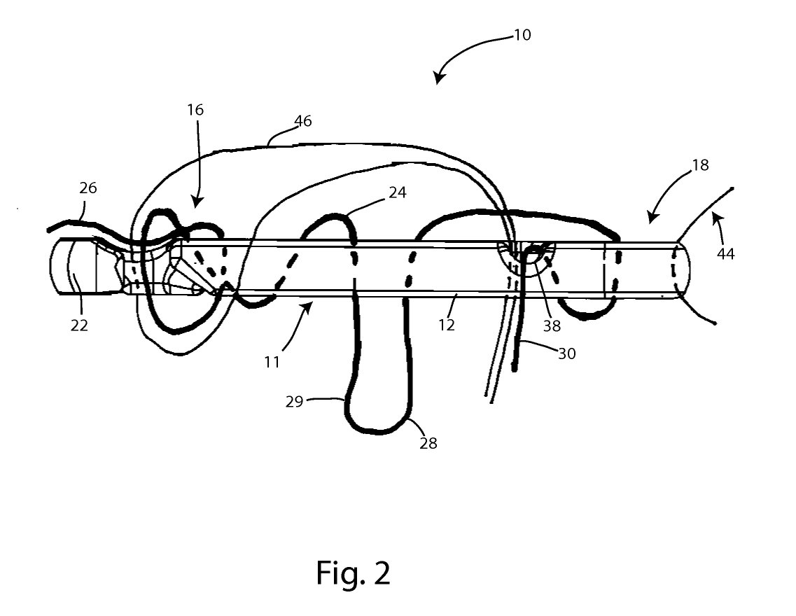

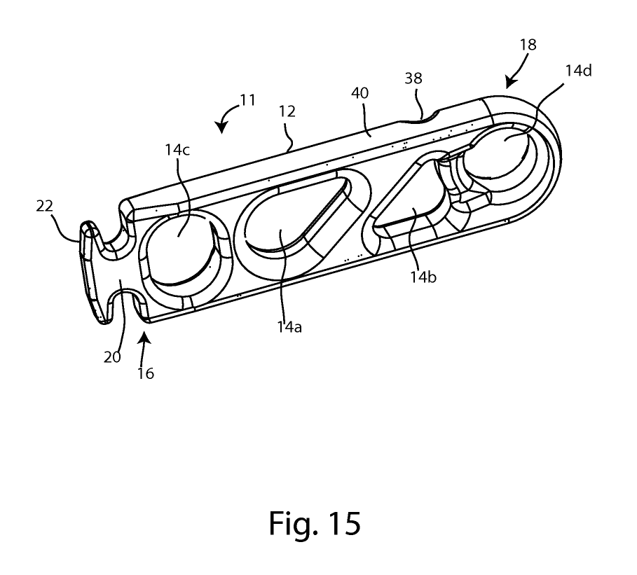

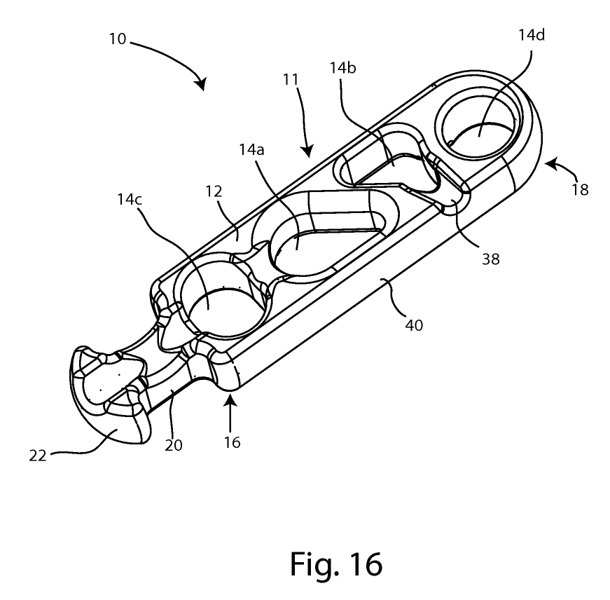

One embodiment is a rectangular piece/plate with rounded ends. One of the ends has a “dogbone” feature for lines and filaments to wrap around. The dogbone feature may comprise two cutouts toward one end of the plate on opposing sides, forming a neck, which give the structure its name as featured in the FIGS. 1-14 and 17-21. FIGS. 15-16 show what may be a dogbone feature, however the dogbone feature may be less pronounced than in the other figures. There may be four holes in the plate; two on each end, and two holes more centrally located in the plate. A line which may be a suture, a cord, or a filament material is passed through the holes to create an adjustable loop/sling to hold an ACL graft or other biological material. The graft line material is fixed to the dogbone end via a looping technique that secures the line to the plate and simultaneously provides a first loop that is configured to be pulled on directly or have a filament passed through this first loop. Such a looping technique may include those as described in U.S. Pat. Nos. 7,594,923, 7,566,339 and 7,150,757 which are herein incorporated by reference. This first loop can be pulled on directly or via secondary filaments to pull or position the plate through a tunnel or hole. A non-looped end, or a free end, of the line material passes around the plate and back under itself to form a self-locking wrap. When the free end of the line is pulled, the adjustable loop, or sling, can be shortened. When the adjustable loop is pulled on, the self-locking wrap cinches itself tighter.

The plate is designed to fit through a bone tunnel and can be inserted via any preferred approach (e.g., medial portal or trans-tibial approaches). A first or primary filament may have a first filament loop created along its length a specified distance from its mid-length, the loop may be stationary but may also move along the length of the first filament. The first filament may have a stationary feature, which may be a knot, at the filament's mid-length. A section of the first filament is looped through the plate body on one side. The secondary filament may pass through the body of the plate or through the first loop of the line. A second filament may be passed through one of the central holes in the body of the plate which may be the same hole that the first loop of the line is passed through. The second or secondary filament may also pass through the first filament loop. The second filament may be fixed to the plate at any specified distance from the central plate hole such as passing the second filament around the dogbone feature of the plate or around the plate in general or through any of the other plate holes. The colors and lengths of the filaments may be configured to ensure the primary filament through the plate hole will be pulled through the bone tunnel to pull up the button past the cortical wall, with the secondary filament parallel with the first loop and graft. Once the plate clears the bone tunnel, the secondary filament is pulled on either trans-tibially or from a medial portal to flip the plate so that the plate is perpendicular to the tunnel and anchors against the bone (plate contact area larger the bone tunnel area). After plate installation one end of the primary filament is pulled through the plate, simultaneously pulling the secondary filament from the bone tunnel and central hole in the body of the plate. The secondary filament is pulled from the plate.

One will appreciate that the secondary filament is not required to pass through a first filament loop and thus the secondary filament may not be removed in conjunction with removal of the primary filament. The secondary filament may pass through the bone tunnel and after installation of the plate the secondary filament may be removed through the plate similar to the primary filament.

This device may be used to hold an ACL graft and the graft may be soft or hard tissue. In addition this device is not limited to the ACL or knee and may have other applications in other parts of the body, such as the shoulder, elbow and ankle.

Referring to FIGS. 1-5, a fixation device 10 is portrayed with a plate 11, a line 24 and filaments 44, 46. The plate 11 may be substantially rectangular and may comprise a body 12 which may have rounded first end 16 and a rounded second end 18. The body may include a plurality of passageways 14 configured to receive the line 24 and first and second filaments 44, 46. The plurality of passageways 14 may be partially or entirely bounded by the plate 11. The plate may also comprise a neck 20 extending longitudinally from the first end 16 of the body 12; however, the neck 20 may extend from either the first end 16 or the second end 18, or both. The neck 20 may be thinner than the body 12 of the plate 11 in at least one plane or the neck 20 may have a smaller circumference than the body 12 of the plate 11. Extending longitudinally from the neck is a head 22 which may be thicker than the neck 20 in at least one plane or may have a greater cross-sectional diameter in at least one plane than the neck 20. The head 22 may also extend from the neck in at least one plane substantially perpendicular to the neck 20. The head 22 may have a substantially similar thickness as the body 12 of the plate or may have a substantially similar cross-sectional diameter in at least one plane as the body 12 of the plate 11. The neck 20 and head 22 of the plate 11 create a dogbone 34 feature on one end of the plate 11. The head 22 may be rounded on the end opposite the neck 20.

The plate 11 may also comprise grooves 36 throughout the plate. The grooves 36 may extend between passageways 14 or between a passageway 14 and a periphery 40 of the plate 11 or even grooves 36 from between the head 22 and neck 20 and the neck 20 and the body 12. The grooves 36 may also reside on the top, sides or bottom of the plate 11. The plate 11 is designed to fit through a bone tunnel and can be inserted via any preferred approach (e.g., medial portal or trans-tibial approaches). The plate may be comprised of biocompatible materials including but not limited to titanium, stainless steel, cobalt chrome, PEEK, PLLA, polymer/ceramic composites, polymers, co-polymers, or alloys or a combination of those mentions herein. In addition any material used for the plate may also be coated with bioactive or supportive materials.

The plurality of passageways 14 may be generally rounded and are capable of receiving at least one line. The plurality of passageways 14 may comprise four passageways that are configured to receive the line 24 and are shaped and patterned for the routing of the line through the passageways 14. The passageways 14 may be substantially on the body 12 of the plate 11; however in an alternate embodiment the neck 20 may also comprise passageway 14. Of the four passageways those disposed more laterally may comprise a more ovoid shape and those passageways disposed more medially may comprise a more triangular or tear-drop shape. The shapes of the plurality of passageways 14 is to enhance the routing and self-locking of the line 24 to the plate 11. The tear-drop shape of at least one of the plurality of passageways 14 may further enhance the locking of the line 24 for the adjustable loop 29. The ovoid shape of at least one of the plurality of passageways 14 may allow for multiple passes of the line 24 through the same passageway while minimizing total passageway area. The plurality of passageways 14 may also taper or enlarge from the top to the bottom of the plate 11 or the plurality of passageways 14 may taper or enlarge from the bottom to the top of the plate 11.

The neck 20 is a smaller circumference than the body 12, or is thinner than the body in at least one plane, to maintain the line 24. The smaller circumference may also provide protection of the lines as they pass through the cutouts that create the neck to prevent the lines from rubbing against the walls of a bone tunnel when passing the plate 11 through the bone tunnel.

The head 22 of the plate 11 may comprise flanges or fins 42 which extend back toward the body 12 of the plate 11. These flanges 42 may add greater security of the line 24 and the second filament 46 preventing withdrawal of the second filament 46 or the line 24 over the head 22. The flanges 42 may also provide added protection of the line 24 and second filament 46 while passage of the plate through the bone tunnel.

The line 24 of the device 10 is routed through the passageways 14 to create a self-locking slide. The line 24 may be comprised of metal, polymer, composite or suture and may be woven or braided. The line may comprise a first portion, which may be a first working portion 26, and a second portion, which may be a second working portion 30. Both of the first and second working portions 26, 30 may have free ends. Between the first working portion 26 and the second working portion 30 is an intermediate portion 28 which may comprise an adjustable loop 29. The first working portion 26 is routed along a first pathway. The first pathway may comprise routing the first working portion up through a first medially located passageway 14a, through at least one of the grooves 36, down through a first laterally located passageway 14c, around the neck 20, up through the first laterally located passageway 14c and passed underneath the portion of the line around the neck 20. The neck 20 may comprise a neck groove 32 that the line passes through underneath the portion of the line 24 that is wrapped around the neck 20. A first compression section 25 is formed with the line passing underneath the portion of the line that passes around the neck 20 wherein when the first working portion 26 is pulled tight the compression section 25 pushes a portion of the line against the neck groove 32 of the neck 20 self-locking the line 24 against the plate 11. This compression section 25 of the line creates a one-way slide allowing for the first working portion 26 to be advanced only along one direction, the one direction defined by the routing of the first working portion 26.

The second working portion 30 is routed along a second pathway. The second pathway may comprise routing the second working portion up through the first medially located passageway 14a, passed over the top of a periphery groove 38, the periphery groove 38 extending from a second medially located passageway 14b to the periphery 40 of the plate. The second working portion 30 is then passed down through the second laterally located passageway 14d, up through the second medially located passageway 14b and underneath the portion of the line 24 that passed over the periphery groove 38. A second compression section 27 is formed with the line passing underneath the portion of the line that passes over the top of the periphery groove 38 wherein when the second working portion 28 is pulled tight the compression section 27 pushes a portion of the line against the periphery groove 38 of the plate 11 self locking the line against the plate 11. This compression section 27 of the line creates a one-way slide allowing for the second working portion 30 to be advanced only along one direction, the one direction defined by the routing of the second working portion 30.

The adjustable loop 29 of the intermediate portion 28 of the line 24 is configured to hold a graft (not shown). The graft may be an ACL graft and may be a soft or hard tissue. The graft is looped around the adjustable loop 29 to retain the graft. By pulling on either the first working portion 26 or the second working portion 30, or both, the adjustable loop 29 reduces in size pulling the graft closer to the plate 11. The adjustable loop 29 is unable to increase in size after the graft is captured by the adjustable loop 29 and the line 24 is routed through the plate 11 because the routing of the line 24 forms a one-way slide so only reduction of the adjustable loop 29 occurs.

The first filament 44 passes through the second laterally positioned passageway 14d and is used to pull the plate 11 through the bone tunnel along a longitudinal axis of the plate 11. The second filament 46 may be routed around the neck 20, creating a loop around the neck 20, of the plate 11 and then passed through the second medially positioned passageway 14b. The second filament 46 is used to toggle the plate 11 after the plate 11 passes through the bone tunnel. After passing through the bone tunnel the second filament 46 may remain in the bone tunnel. The second filament 46 is pulled and the plate 11 toggles so that the longitudinal axis of the plate 11 is perpendicular to the bone tunnel. After the plate 11 is positioned on a cortical side of a bone the first and second filaments 44, 46 may be removed by pulling on one end of each filament. After positioning of the plate 11 the first working portion 26 or the second working portion 30, or both, are pulled to reduce the size of the adjustable loop 29 thereby creating greater tension in the graft and further cinching the plate 11 against the cortical side of the bone.

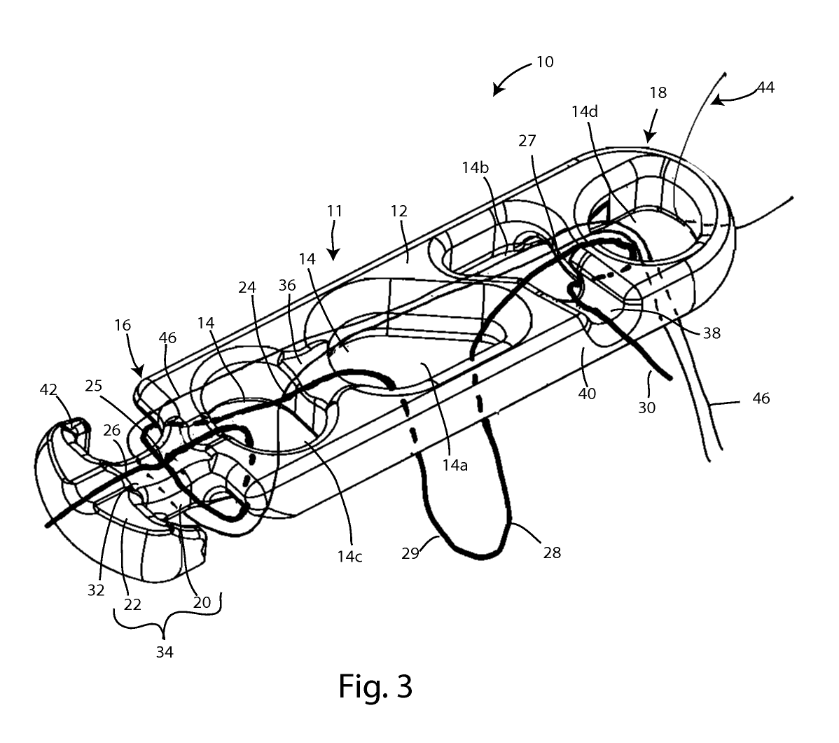

The routing of the second filament 46 may be done in a plurality of ways. Referring to FIG. 3, the second filament 46 may be routed around the neck 20, creating a loop around the neck 20, similar to the embodiment in FIG. 1. The second filament 46 then passes through the second laterally positioned passageway 14d. The same effect is achieved with this routing as that previously described in that the second filament 46 is used to toggle the plate 11 after passing through the bone tunnel. Similar to the previous routing, after passing through the bone tunnel the second filament 46 may remain in the bone tunnel. After the plate 11 is positioned on the cortical side of the bone the second filament 46 is removed.

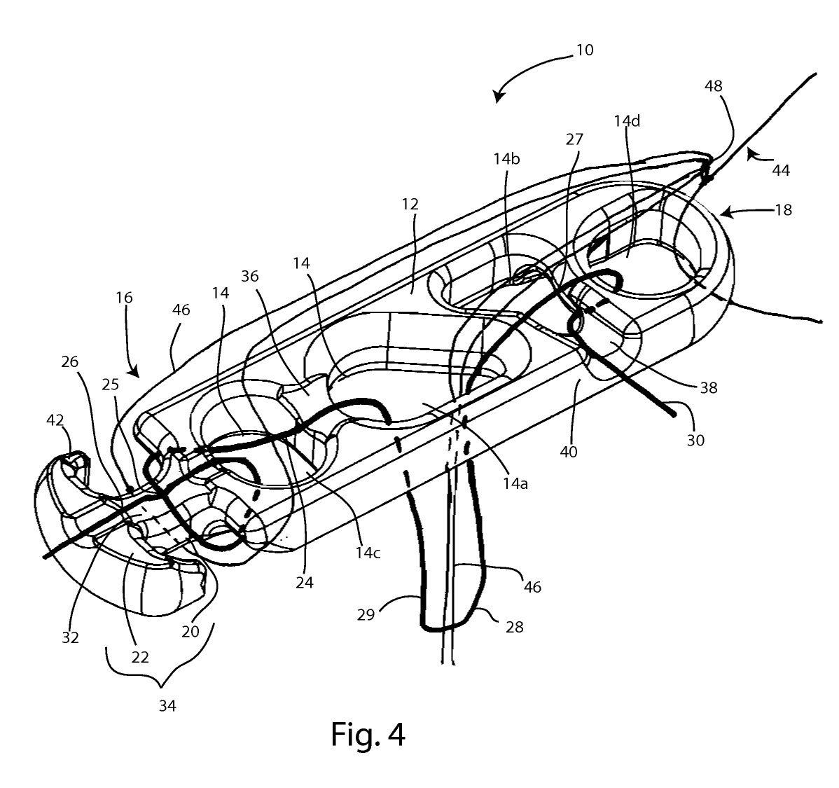

Referring to FIG. 4, the second filament 46 may be routed around the neck 20, creating a loop around the neck 20, and then the second filament passed slidably through a first filament loop 48 of the first filament 44. The first filament loop 48 may be static. The second filament 46 may then be routed through any of the plurality of passageways 14. After passage of the plate 11 through the tunnel the second filament 46 is again used to toggle the plate 11 so that the longitudinal axis of the plate is perpendicular to the bone tunnel. Similar to the previously described routing, after passing through the bone tunnel the second filament 46 may remain in the bone tunnel. After positioning of the plate 11, the first filament 44 may be removed thereby removing the second filament 46 as well because the second loop is still within the first filament loop 48.

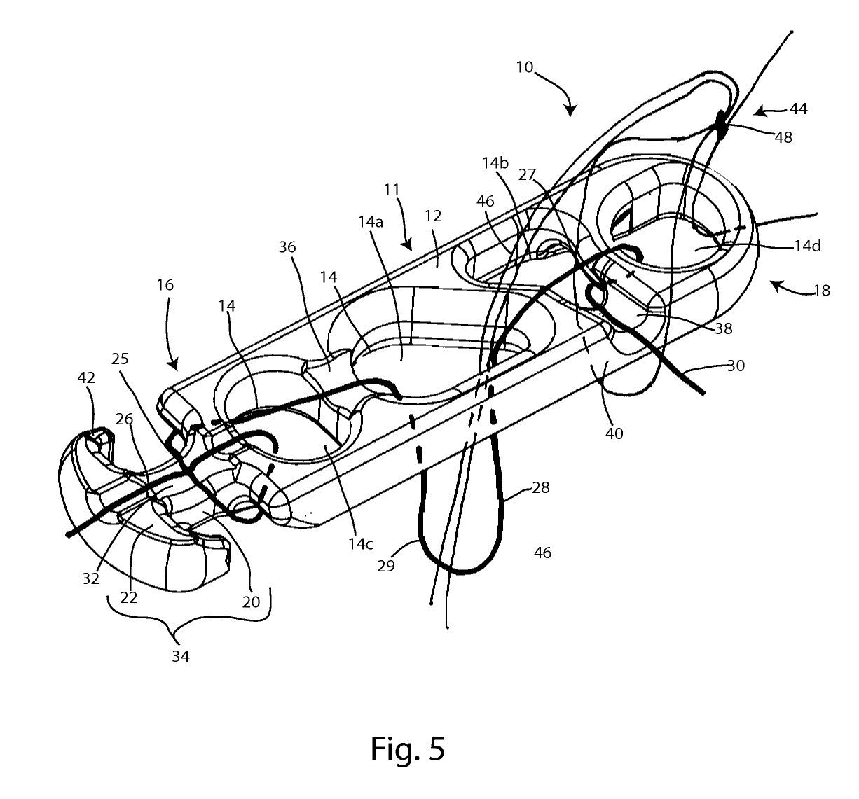

Referring to FIG. 5, the second filament 46 may be routed through any of the plurality of passageways 14 creating a loop around the intended passageway. The filament is then routed through the first filament loop 48 of the first filament 44. The second filament 46 may then be routed through any of the plurality of passageways 14. Once the plate 11 clears the bone tunnel, the second filament 46 is pulled on (trans-tibially or from the medial portal) to flip the plate so that the plate 11 is perpendicular to the bone tunnel and anchors against the bone with the plate contact area being larger than the bone tunnel. Similar to the previously described routing, after passing through the bone tunnel the second filament 46 may remain in the bone tunnel. Similar to the removal of the first filament 44 in the preceding paragraph, after positioning of the plate 11, the first filament 44 may be removed thereby removing the second filament 46 as well because the second loop is still within the first filament loop 48.

After the plate 11 is secured against the cortical bone, the filaments 44, 46 are removed and the plate 11 is cinched and the adjustable loop 29 adjusted to the appropriate length and tension the free ends of the first and second working portions 26, 30 may be cut to shorter lengths.

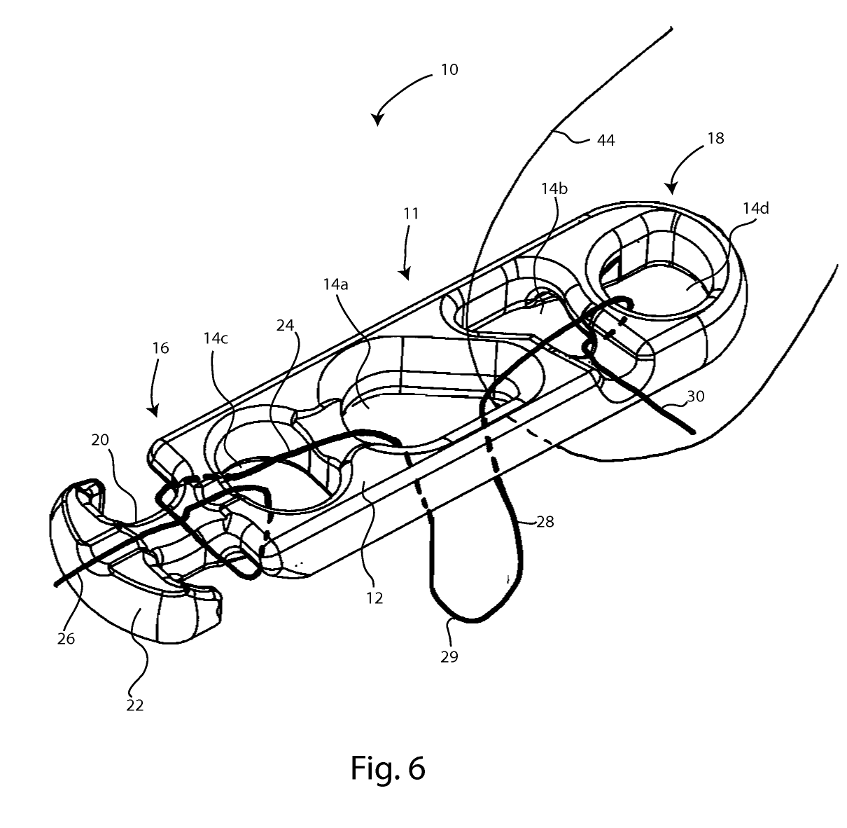

Referring to FIG. 6, all of the features previously disclosed are substantially the same; however only a one filament, the first filament 44, is used and passed through the more centrally located first medial passageway 14a. The first filament 44 passes through the first medial passageway 14a. The plate 11 is inserted in a longitudinal direction, substantially parallel to the axis of the bone tunnel, into the bone tunnel. Pulling on the first filament through the bone tunnel the plate 11 passes through the bone tunnel and after clearing the cortical side of the bone the plate 11 toggles automatically, wherein the longitudinal direction of the plate is substantially perpendicular to the axis of the bone tunnel. Because of the placement of the first filament 44 through the first medial passageway 14a, the plate 11 naturally tends to return to a non-parallel state between the plate 11 and the bone tunnel. In this embodiment a second filament is not needed to toggle the plate 11.

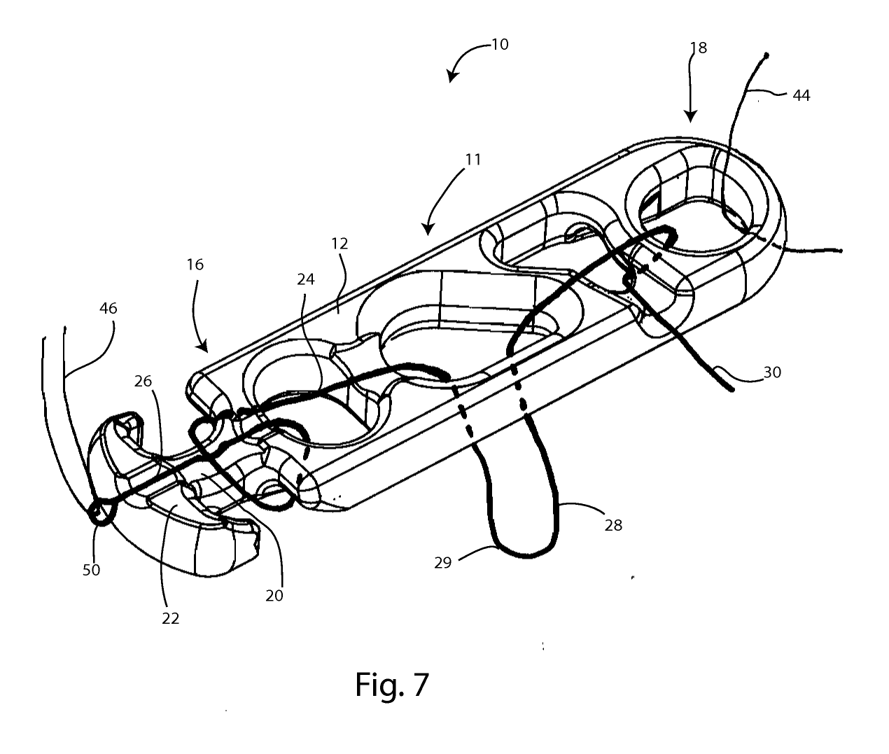

Referring to FIG. 7, all of the features previously disclosed are substantially the same to this embodiment with the exception of the first working portion 26 may comprise a first loop 50 instead of a free end. The first loop 50 may be an eyelet. The first loop 50 may be woven or braided into the cord or the first loop 50 may be a thicker cord with separate fibers of the cord. The first loop 50 may also use an adhesive in the fibers to help maintain the structure of the loop. The first loop 50 may also be created by using the free end of the first working portion 26 and crimping the free end with a crimp (not shown) to another portion of the first working portion 26 to create a loop. The crimp could be metal, polymer or any other biocompatible materials strong enough to hold the line 24 to form the first loop 50. The first loop 50 may be held tightly against the plate 11 or may be kept at a distance from the plate 11.

The first loop 50 may be configured to receive the second filament 46. The second filament 46 is looped through the first loop 50 and is used to toggle the plate 11 after passage through the bone tunnel. The second filament 46 through the first loop 50 provides certain advantages when pulling the plate 11 through the bone tunnel including increased slidability of the second filament 46 and may provide an offset distance from the plate 11 to improve flipping of the plate 11. Furthermore the first loop 50 may allow for slidability of the second filament 50 preventing the second filament 46 from getting stuck or damaged between the plate 11 and the bone.

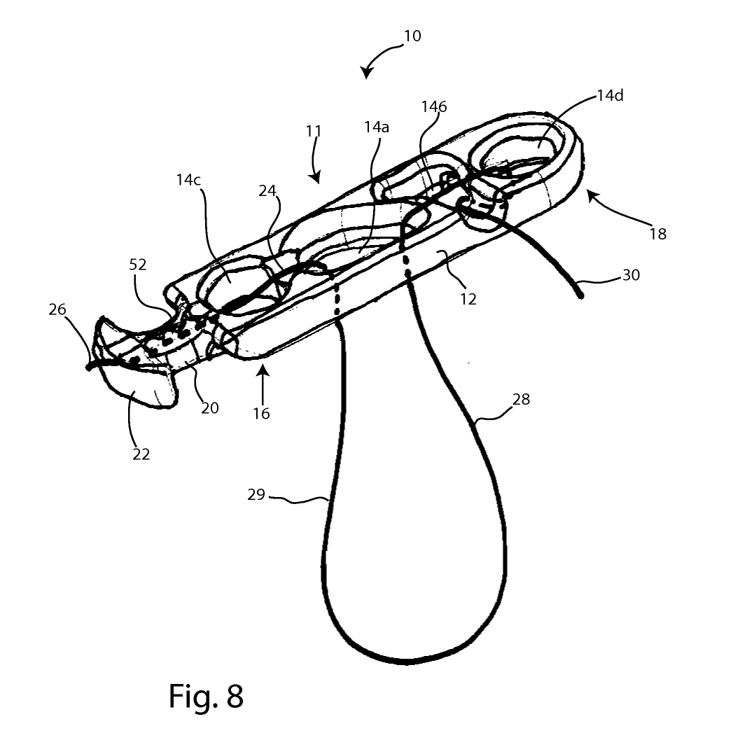

Referring to FIG. 8, a device 10 is depicted with a slight different head 22 extending from the neck 20. In this embodiment the head 22 may not include the flanges or fins 42 as depicted in the previous embodiments (FIGS. 1-7). Furthermore, this embodiment may include a crimp 52 on the dogbone end 34, wherein the first working portion 26 of the line 24 passes through the neck groove 32 and is secured to the plate 11 on the neck 22 or the dogbone end 32 through the use of the crimp 52. The crimp 52 would substantially encircle the neck 20 and the first working portion 26 of the line 24 to the plate 11. The crimp 52 may be comprised of any biocompatible material including polymer, composite or metal. A further distinguishing feature of this embodiment is that the first working portion 26 may or may not be routed in the same or similar manner as the routing in the previous embodiment (FIGS. 1-7). With the FIG. 8 embodiment the first working portion 26 may simply pass up through the first medial passageway 14a but may not pass completely back down and through the first lateral passageway 14c, but rather simply pass through the neck groove 32 and then be secured to the plate 11 with the crimp 52. The second working portion 30 of the line 24 is routed in substantially the same manner as described in the previous embodiments described in FIGS. 1-7 with the same routing of the line 24, the same adjustable loop 29 of the intermediate portion 28 and the second compression section 27 which holds a portion of the line 24 to create a one-way slide.

First and second filaments 44, 46 (not shown in FIG. 8) may be routed around and through the plate 11 in any manner as previously described herein. Likewise inserting the plate 11 into the bone tunnel, passing the plate 11 through the bone tunnel, toggling the plate 11 after passage through the bone tunnel, cinching the plate to the cortical side of the bone and removing the filaments 44, 46 from the plate may all be accomplished in any of the ways as previously described herein.

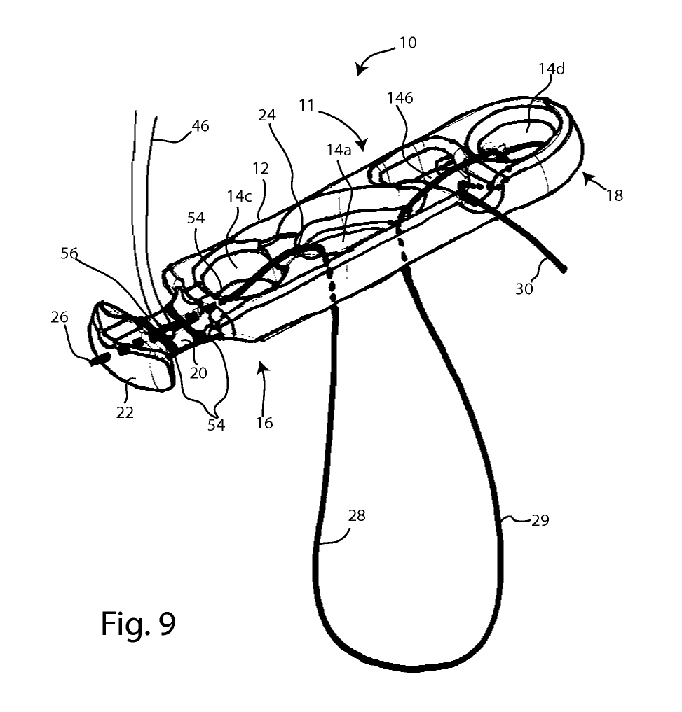

Referring to FIG. 9, a device 10 is depicted with the nearly the same plate 11 configuration as that of FIG. 8; however, in this embodiment there is a neck hole 54 that passes from the first lateral passageway 14c through the neck 20 and out of the head 22. The neck 20 may include an opening with two transverse pins 54. The first working portion 26 of the line 24 may pass through the neck hole 54 wherein a portion of the first working portion 26 is exposed in a neck opening 56. The neck opening 56 may be defined by two transverse pins 54 that are substantially perpendicular to the longitudinal axis of the plate 11. The transverse pins 54 may retain the line 24 and prevent it from withdrawal from the plate 11. The first filament 44 (not shown in FIG. 9) may be passed through at least one of the plurality of passageways 14. The second filament 46 may pass around the exposed portion of the first working portion 26 in the neck opening 56 and is retained by the exposed portion of the first working portion 26. The first filament 44 (not shown in FIG. 9) may be passed through at least one of the plurality of passageways 14. The second working portion 30 of the line 24 is routed in substantially the same manner as described in the previous embodiments described in FIGS. 1-7 with the same routing of the line 24, the same adjustable loop 29 of the intermediate portion 28 and the second compression section 27 which holds a portion of the line 24 to create a one-way slide.

A method of inserting the plate 11 into the bone tunnel, passing the plate 11 through the bone tunnel, toggling the plate 11 after passage through the bone tunnel, cinching the plate 11 to the cortical side of the bone and removing the filaments 44, 46 from the plate may be accomplished in any of the ways as previously described herein.

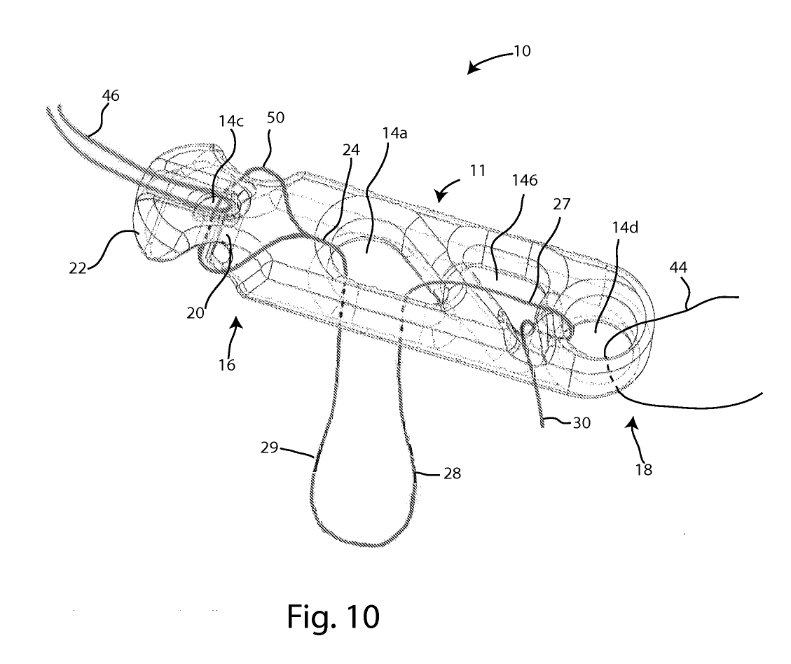

Referring to FIG. 10, a device 10 is depicted with the plurality of passageways 14 as previously described; however, in this embodiment one of the plurality of passageways is positioned in the neck 20 of the plate 11. The second working portion 30 of the line 24 is routed in substantially the same manner as described in the previous embodiments described in FIGS. 1-7 with the same routing of the line 24, the same adjustable loop 29 of the intermediate portion 28 and the second compression section 27 which holds a portion of the line 24 to create a one-way slide. The first working portion 26 of the line 24 includes the first loop 50 as described in FIG. 7. In this embodiment the first loop 50 is not secured to the plate through a first compression section 25 as previously described; rather, the first loop 50 is lassoed around the neck 20. The second filament 46 is then able to pass through the passageway in the neck, the first lateral passageway 14c, and loop around the first loop 50, the first loop 50 retaining the second filament. The first filament 44 is passed through the second lateral passageway 14d and the filaments 44, 46 are used to pass the plate 11 and toggle the plate 11 in any manner as previously described herein. It will be appreciated that the first filament 44 may pass through any of the remaining plurality of passageways 14 of the plate 11 except for the first lateral passageway 14c.

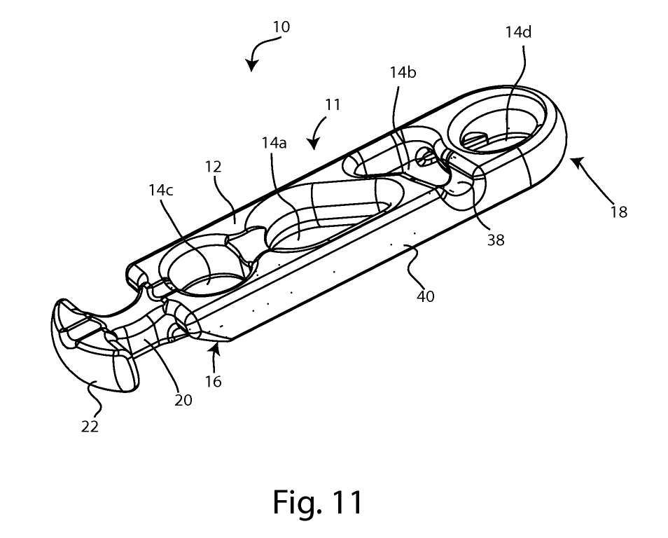

Alternate embodiments of the plate are depicted in FIGS. 11-21. In each of the FIGS. 11-21 the line 24 has not been shown neither do the figures show the first and second filaments 44, 46. It should be understood that the line 24 and filaments 44, 46 may be used for those embodiment described and illustrated for FIGS. 11-21 in the same manner as any previously disclosed embodiment in the paragraphs and illustrations above. For the avoidance of doubt, the line may comprise two free ends or one free end and a loop or further still one free end and another securing means as described above such as crimps, pins, loops or clamps. In addition each of these embodiments may comprise a looped end on the first working portion 26 and a free end on the second working portion 30 as set forth above.

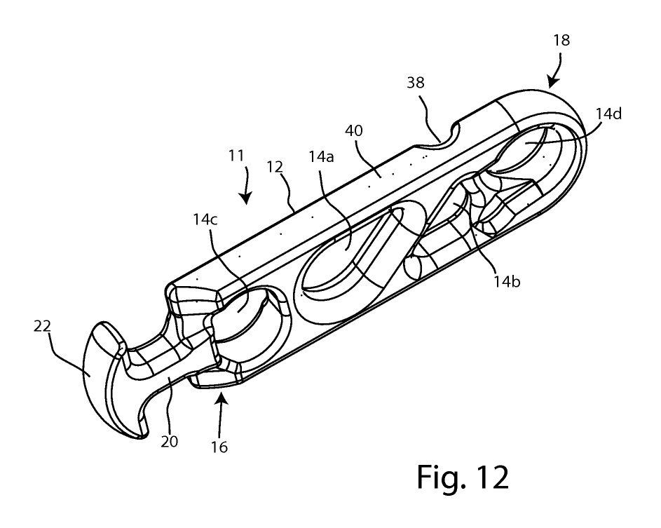

Referring to FIGS. 11-12, the plate 11 is the same as shown in FIGS. 8 and 9 without the line 24 and the first and second filaments 44, 46 depicted. The line 24 and filaments 44, 46 may be used in this embodiment in any of the previously described methods found herein.

The features in this embodiment are very similar to the previous embodiment of the plate in FIGS. 1-7 with the exception that the plurality of passageways 14 may have slightly different shapes where the substantially triangular or tear-drop shaped passageways may have other bends and the ovoid passageways may be more circular. Other distinguishing features of this embodiment of the plate 11 are the shape and features of the head 22 of the dogbone 34 feature. Like the previous embodiment the head 22 has a greater cross-sectional diameter than the neck 20 in at least one plane. Whereas the previous embodiment may have comprised flanges or fins 42 extending back toward the body 12 of the plate 11 giving the dogbone feature 34 an almost anchor like look, this embodiment does not require the fins 42 but rather the head 22 may only extend outward from the neck 20 in at least one plane perpendicular to the longitudinal axis of the plate 11. The head 22 may also be rounded on one end opposite from the neck 20. The same features found in the previous embodiment hold true for this embodiment in that there are grooves 36 which may pass between the plurality of passageways and this embodiment may also include a periphery groove 38 extending from one of the second medially passageway 14b to the periphery 40 to receive the second working portion 30 of the line 24.

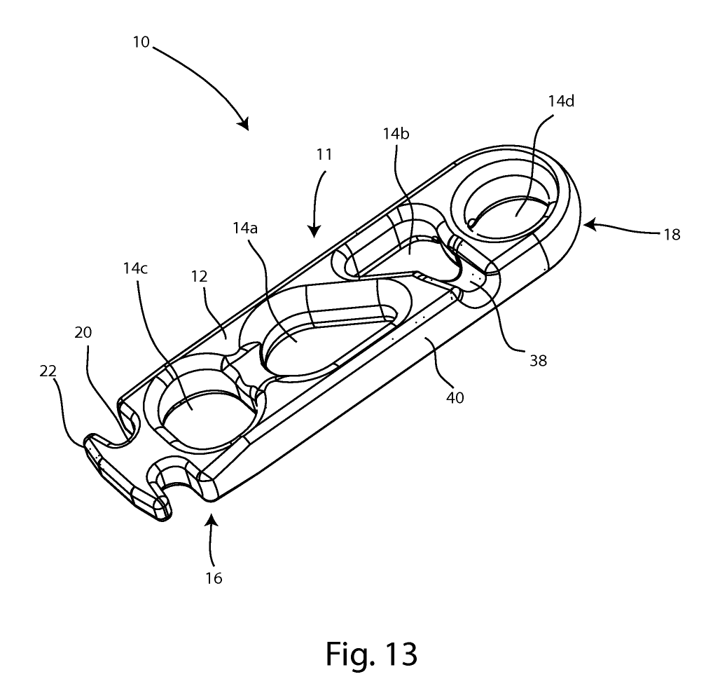

Referring to FIGS. 13-15, the plate 11 may include similar features as the previously described plates with passageways and grooves; however, in this embodiment the neck 20 may be shorter than in the previous embodiments and the head 22 extending from the neck 20 creating the dogbone feature 34 has previously set forth. The head 22 may have a larger circumference than the neck 20 but smaller than the body 12 of the plate 11.

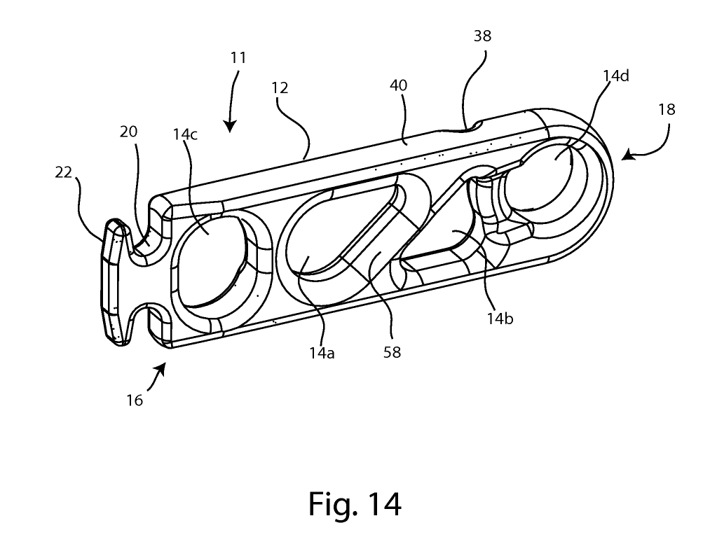

Referring to FIG. 14, the plurality of passageways 14 may comprise curved ends 58 that allow for easy passage of the line 24 through the plurality of passageways 14. The curved ends 58 of the plurality of passageways 14 may extend into each other as depicted between the first medial passageways 14a and second medial passageway 14b. Alternately, as depicted in FIG. 15 between the first medial passageways 14a and second medial passageway 14b, the curved ends 58 may be separated by part of the body 12 of the plate 11.

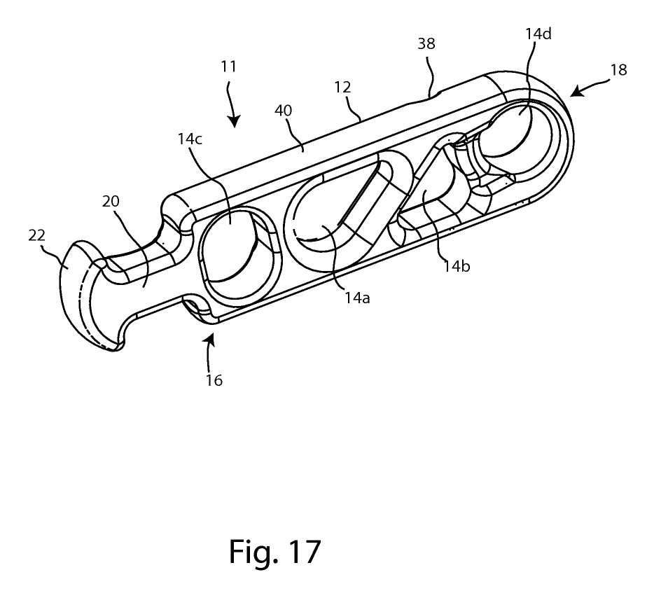

Referring to FIGS. 16-17, this alternate plate 11 embodiment comprises substantially all of the features as previously described herein with a neck 20 and head 22 creating a dogbone feature 34. The neck may have a substantially smaller cross-sectional diameter than the body 12 of the plate 11 and the head 22 may have a larger circumference than the neck 11; however the head 22 may have a smaller cross sectional diameter in at least one plane than the body 12 of the plate 11. The shapes of the plurality of passageways 14 may be substantially the same as the previous embodiments with the lateral passageways 14c, 14d maintaining a substantially ovoid or circular shape and the medial passageways 14a, 14b maintaining a substantially triangular or tear-drop shape.

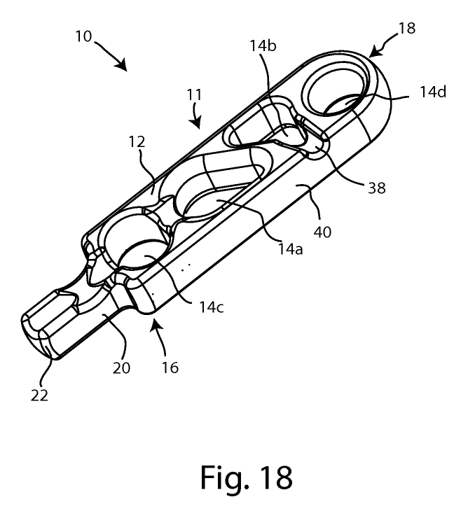

Referring to FIG. 18, this alternate embodiment of the plate 11 may comprise features substantially similar to those already described herein. However, the neck 20 and head 22 may of the dogbone feature 34 may have substantially the same cross-sectional diameter in at least one plane. The neck 20 extends from at least one end of the body 12 of the plate 11 and the head 22 extends from the neck 20 but the extension may look like a singular post extending from the body 12 of the plate 11 rather than a dogbone feature 34 as previously recited. The use of this embodiment remains the same in that the routing of the line 24 and the use of the first and second filaments 44, 46 remains may be used in any of the previously described methods.

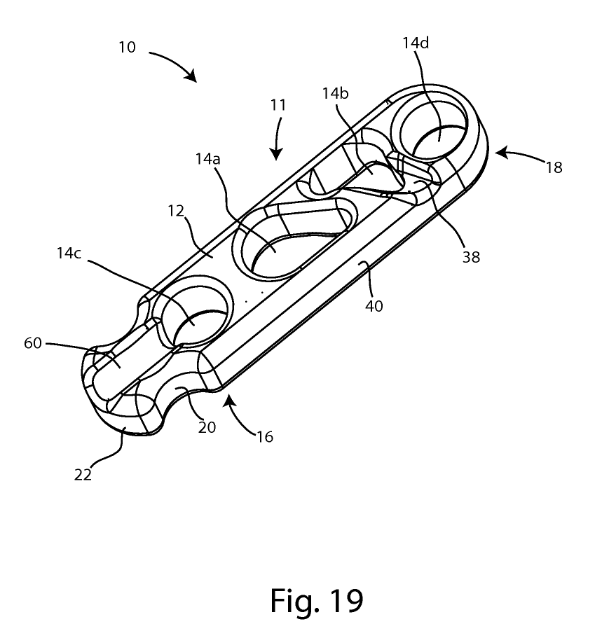

Referring to FIG. 19, this alternate embodiment of the plate 11 may comprise features substantially similar to those already described herein. In this embodiment, however, the neck 20 and head 22 may comprise a deep groove 60 extending from one end of the head 20 to the first lateral passageway 14c. The deep groove 60 may provide easier passage of the first working portion 26 of the line 24 underneath the compression section 25.

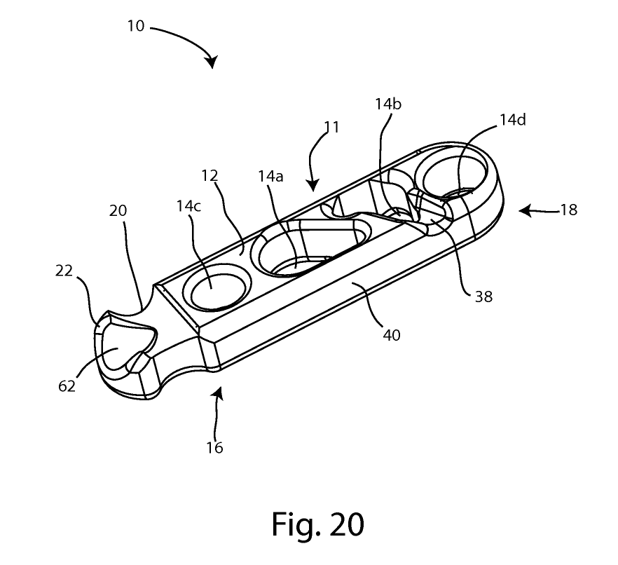

Referring to FIG. 20, this alternate embodiment of the plate 11 may comprise features substantially similar to those already described herein. However, a cutout 62 may extend from the head 22 toward the neck 20. The cutout 62 may provide easier passage of the first working portion 26.

Other characteristics which are not depicted in the figures may include other means of securing the second working portion of line in addition to the use of the compression section and routing of the lines to create a one-way slide. On the end opposite the dogbone feature of the plate a slot may extend from the opposite end into the body of the plate where in the slot gets wider as it moves further from the periphery of the plate. The slot may be configured to receive and pinch a line keeping the line substantially static after the appropriate length and tension of the line is determined. Other means for additional security may be the use of a cleat or locking feature extending from the plate opposite the dogbone feature. The cleat may be used to tie off the free end of the line after the appropriate length and tension of the line is determined.

All of the embodiments illustrated and described herein may have features mixed and matched to create a plate of physician's choice. The plurality of passageways 14 may be spaced apart at greater or lesser distance from one another. Similarly the plurality of passageways 14 may reside nearer or further from the periphery 40 of the plate 11. Each of the plurality of passageways 14 may be smaller or larger so long as they are capable of receiving at least one line and/or filament.

The present invention may be embodied in other specific forms without departing from its spirit or essential characteristics. For example, above are described various alternative examples of plates and securing of lines as well as routing of the line and the routing of filaments. It is appreciated that various features of the above-described examples can be mixed and matched to form a variety of other combinations and alternatives. It is also appreciated that this system should not be limited to simply ACL repair and fixation. This system may also be used to secure other ligaments, tendons or soft or hard tissue. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for retaining a graft, the system comprising:
a plate comprising;
a body formed of biocompatible material, the body having an elongated shape and at least one end, the body at least partially bounding a plurality of passageways;
a neck extending from the at least one end of the body, the neck thinner than the body in at least one plane;
a head extending from the neck, the head is thicker than the neck in at least one plane; and
a continuous line routed through the plurality of passageways of the body, the line comprising a first portion, an intermediate portion, and a second portion;
wherein the first portion comprises a first loop, the intermediate portion comprises a second adjustable loop, and the second portion comprises a free end.

2. The system of claim 1, wherein the neck and head comprise a dogbone feature extending from the at least one end of the body of the plate.

3. The system of claim 1, wherein a first groove extends longitudinally from the head to the neck to one of the plurality of passageways of the body of the plate.

4. The system of claim 1, wherein the first portion comprises a first working portion and the second portion comprises a second working portion and a compression section wherein the second portion presses the compression section against the body providing a self-locking one-way slide.

5. The system of claim 1, wherein the plurality of passageways comprise four substantially bounded passageways that cooperate to receive the first and second portions.

6. The system of claim 5, wherein a second groove extends between a first passageway and a second passageway of the plurality of passageways.

7. The system of claim 6, wherein a third groove extends from a third passageway to a periphery of the body of the plate.

8. The system of claim 7, wherein a fourth groove extends from the third passageway to a fourth passageway.

9. The system of claim 1, wherein the second adjustable loop is configured to retain a graft.

10. The system of claim 1, wherein at least one of the plurality of passageways is configured to receive a first filament wherein the first filament is routed through at least one of the plurality of passageways of the body.

11. The system of claim 1, wherein the first loop of the line is configured to receive a second filament, wherein the second filament is routed through the first loop.

12. The system of claim 1, wherein the second adjustable loop is adjustable in only one direction to reduce the size of the loop.

13. The system of claim 1, wherein a first filament is routed through at least one of the plurality of passageways of the body and a second filament is routed through the first loop.

14. The system of claim 1, wherein a first filament passes through at least one of the plurality of passageways of the body of the plate, the first filament comprising a first filament loop and a second filament passes around the neck, passes slidably through the first filament loop and passes through at least one of the plurality of passageways of the body of the plate.

15. The system of claim 1, wherein a first filament is routed through at least one of the plurality of passageways of the body and the second filament is routed through at least one of the plurality of passageways of the body.

16. A system for retaining a graft ligament, the system comprising:
a plate comprising;
a body formed of biocompatible material, the body having an elongated shape, the body at least partially bounding a plurality of passageways;
a neck extending from at least one end of the body, the neck thinner than the body in at least one plane;
a head extending from the neck; and
a continuous cord routed through the plurality of passageways of the body, the line comprising a first portion, an intermediate portion, and a second portion;
wherein the first portion comprises a first free end, the intermediate portion comprises an adjustable loop and the second portion comprises a free end:
wherein the first portion is routed at least partially around the neck to secure the cord to the neck.

17. The system of claim 16, wherein the head is thicker than the neck in at least one plane.

18. The system of claim 17, wherein the neck and head comprise a dogbone feature extending from the body of the plate.

19. The system of claim 16, wherein a first groove extends longitudinally from the head to the neck to one of the plurality of passageways of the body of the plate.

20. The system of claim 16, wherein the first portion comprises a first working portion and a first compression section and the second portion comprises a second working portion and a second compression section wherein the first working portion presses the first compression section against the body providing a first self-locking one-way slide and second working portion presses the second compression section against the body providing a second self-locking one-way slide.

21. The system of claim 16, wherein the first working portion comprises a soft loop.

22. The system of claim 16, wherein the plurality of passageways comprises four substantially bounded passageways that cooperate to receive the first and second working portions.

23. The system of claim 22, wherein a second groove extends between a first passageway and a second passageway of the plurality of passageways.

24. The system of claim 22, wherein a third groove extends from a third passageway to a periphery of the body of the plate.

25. The system of claim 16, wherein the adjustable loop is configured to retain a graft.

26. The system of claim 16, wherein at least one of the plurality of passageways is configured to receive a first filament wherein the first filament is routed through at least one of the plurality of passageways of the body.

27. The system of claim 16, wherein the first loop of the line is configured to receive a second filament, wherein the second filament is routed through the first loop.

28. The system of claim 16, wherein the second adjustable loop is adjustable in only one direction to reduce the size of the loop.

29. The system of claim 16, wherein a first filament is routed through at least one of the plurality of passageways of the body and a second filament is routed through the first loop.

30. The system of claim 16, wherein a first filament passes through at least one of the plurality of passageways of the body of the plate, the first filament comprising a first filament loop, and a second filament passes around the neck, passes slidably through the first filament loop and passes through at least one of the plurality of passageways of the body of the plate.

31. The system of claim 16, wherein a first filament is routed through at least one of the plurality of passageways of the body and the second filament is routed through at least one of the plurality of passageways of the body.

32. A method of passing a plate coupled to a graft through a bone tunnel, the method comprising:
looping a graft ligament through a line, the line comprising an adjustable loop;
coupling a first portion of the line to a first end of a body of the plate by the routing of the first portion along a first pathway through a plurality of passageways of the body, the first working portion comprising a first loop, wherein the body of the plate has an elongated shape;
coupling a second portion of the line to a second end of the body of the plate;
routing a first filament through the body of the plate, the body at least partially bounding a plurality of passageways;
routing a second filament through the first loop of the first portion of the line;
inserting the plate into a bone tunnel;
pulling the plate through the bone tunnel along a longitudinal axis of the plate by pulling the first filament through the bone tunnel; and
toggling the plate by pulling on the second filament so the longitudinal axis of the plate is perpendicular to the bone tunnel.

33. The method of claim 32, further comprising passing the second portion along a second pathway creating a one way slide for cinching the plate against a bone, wherein the second portion further comprises a second working portion and a compression section.

34. The method of claim 33, further comprises reducing the size of the adjustable loop of the line by pulling the second portion along the second pathway.

35. The method of claim 32, further comprises routing the first portion around a neck extending from at least one end of the body, the neck is thinner than the body in at least one plane, wherein the first portion further comprises a first working portion.

36. The method of claim 32, further comprises removing the first filament from the plate.

37. The method of claim 36, further comprises removing the second filament from the plate and the bone tunnel.

38. A method of passing a plate coupled to a graft through a bone tunnel, the method comprising:
looping a graft ligament through a line, the line comprising an adjustable loop;
coupling a first portion of the line to a first end of a body of the plate by the routing of the first portion along a first pathway through a plurality of passageways of the body, wherein the body of the plate has an elongated shape;
coupling a second portion of the line to a second end of the body of the plate;
routing a first filament through at least one of the plurality of passageways of the body of the plate;
routing a second filament through at least one of the plurality of passageways of the body of the plate;
inserting the plate into a bone tunnel;
pulling the plate through the bone tunnel along a longitudinal axis of the plate by pulling the first filament through the bone tunnel; and
toggling the plate by pulling on the second filament so the longitudinal axis of the plate is perpendicular to the bone tunnel.

39. The method of claim 38, further comprises pulling the first portion along the first pathway creating a one way slide for cinching the plate against the bone, wherein the first portion further comprises a first working portion and a first compression section.

40. The method of claim 39, further comprises reducing the size of the adjustable loop of the line by pulling the first portion along the first pathway.

41. The method of claim 40, further comprises passing the second portion along a second pathway creating a one way slide for cinching the plate against a bone, wherein the second portion comprises a second working portion and a second compressions section.

42. The method of claim 41, further comprises reducing the size of the adjustable loop of the line by pulling the second portion along the second pathway.

43. The method of claim 38, further comprises routing the second filament around a neck extending from at least one end of the body, the neck is thinner than the body in at least one plane.

44. The method of claim 38, further comprises slidably passing the second filament through a first filament loop of the first filament.

45. The method of claim 44, further comprises preserving the second filament within the bone tunnel.

46. The method of claim 45, further comprises removing the first filament from the plate.

47. The method of claim 46, further comprises removing the second filament from the plate and the bone tunnel by removal of the first filament.
